(19) United States
(12) Patent Application Publication
(10) Pub. No.: US 2025/0262338 A1
(43) Pub. Date: Aug. 21, 2025

(54) APPARATUS, SYSTEM, AND METHOD FOR DISINFECTING AND SANITIZING OBJECTS USING OZONE AND HUMIDITY

(71) Applicant: ZScience, LLC, Johns Creek, GA (US)

(72) Inventors: James Patton, Anderson, SC (US); Walter Mann, Sandy Springs, GA (US); Joseph Patton, Anderson,, SC (US)

(73) Assignee: ZScience, LLC, Johns Creek, GA (US)

(21) Appl. No.: 19/059,601

(22) Filed: Feb. 21, 2025

Related U.S. Application Data
(60) Provisional application No. 63/556,266, filed on Feb. 21, 2024.

(51) Int. Cl. A61L 2/20; A61L 2/06; A61L 2/24; A61L 2/26

(52) U.S. Cl. CPC A61L 2/202; A61L 2/06; A61L 2/24; A61L 2/26; A61L 2202/11; A61L 2202/122; A61L 2202/14

(57) ABSTRACT

The subject matter of the present disclosure relates, in various embodiments, to an apparatus, system and method that provides a new and improved ozone treatment chamber that effectively and efficiently disinfects and sanitizes a multitude of target objects using locally generated ozone and humidity. The present invention comprises a housing unit, an ozone generator, a humidity generator, an ozone destructor and a control system for managing same. In alternate embodiments, in addition to the above structures, a heating system is provided to assist with drying if needed and/or to eradicate lice, bedbugs and their eggs. In use, the present invention disinfectants (at least a 3 log 10 or 99.9% reduction in virus titers) and sanitizes (at least a 3 log 10 or 99.9% reduction in bacterial count) a plurality of viruses and bacteria, respectively, thus eliminating the need for harsh chemical liquids.

### PRIORITY CLAIM

To the fullest extent permitted under applicable law, the present application claims priority to and benefit of U.S. Provisional Patent Application 63/556,266, filed on Feb. 21, 2024, entitled “Apparatus, System and Method for Disinfecting and Sanitizing Objects Using Ozone and Humidity,” and said provisional patent application is incorporated herein by reference.

### TECHNICAL FIELD

The subject matter of the present disclosure relates, generally, to apparatuses, systems, and methods for disinfecting and sanitizing various objects. More particularly, the subject matter of the present disclosure relates to an apparatus, system, and method for disinfecting and sanitizing objects using ozone and humidity.

### BACKGROUND

Worldwide, bacteria and viruses cause an enormous economic, health, and emotional burden on society. More specifically, some of the most common and dangerous bacteria include Staphylococcus aureus (including MSRA), Salmonella enterica, Escherichia coli (E-coli), Streptococcus pyogenes, Shigella dysenteriae, and Pseudomonas aeruginosa. Bacterial infections, collectively, are considered one of the leading causes of death worldwide, resulting in approximately 7.7 million deaths per year and billions of dollars in health costs. Viruses, such as Covid-19, respiratory syncytial virus (RSV), norovirus, and influenza, likewise, also result in millions of deaths per year and billions of dollars in health costs. Not only are these viruses and bacteria an enormous burden to the healthcare system, they are also a tremendous burden to society due to lost productivity that results from the millions of people infected per year. Even more devastating, subjectively, is the enormous emotional and financial burden to the families adversely affected by viruses and bacteria.

One of the most common ways that viruses and bacteria can spread is via porous, non-porous, and semi-porous surfaces. Ultimately, this means that any object could potentially be a carrier for viruses and bacteria. In fact, depending on the type of surface, some viruses/bacteria may survive as little as a few hours, while others could survive as long as several months. For example, on dry non-porous hard surfaces, the influenza virus can survive up to 24 hours; whereas, the Norovirus could survive up to several days; and, whereas, Shigella dysenteriae could survive as long as several months. Ultimately, any environment wherein objects are in contact with humans can serve as a potential vessel for transmitting viruses and bacteria. Moreover, high-use environments having repeated multiple human interaction, such as childcare facilities, schools, sports, fitness facilities, and public safety departments, are at an even higher risk of spreading viruses and bacteria. For example, some studies have shown that more than 70% of toys in a childcare facility can be contaminated with potentially harmful bacteria and/or viruses. Consequently, to minimize these risks, there is a critical need to regularly disinfect and sanitize the various target items utilized in these environments.

Historically, harsh liquid chemicals such as chlorine, chlorine compounds, formaldehyde, glutaraldehyde, ortho-phthalaldehyde, alcohols, hydrogen peroxide, iodophors, peracetic acid, phenolics, and quaternary ammonium compounds have been utilized to sanitize and disinfect these at-risk objects. However, there are many disadvantages in using these harsh liquid chemicals. For example, most of these chemicals, especially after repeated use, will adversely affect the target object's structural integrity; thus, reducing its intended lifespan; and thereby, resulting in increased costs to replace the object. Additionally, because many of these liquid chemicals are harmful to both the person applying the chemical and to the end user, these chemicals must be utilized in lower concentrations, making them potentially less effective; and/or they must be rinsed/wiped; thus, resulting in additional waste by-products. Furthermore, even after rinsing and wiping, the use of these chemicals often results in a residue and strong unpleasant odor remaining on the target item. Moreover, the process of adequately sanitizing and disinfecting target items with these harsh chemicals is extremely labor intensive; thereby, resulting in additional labor costs and added exposure to personnel. Additionally, many of the chemicals are considered hazardous by OSHA and/or a hazardous waste by the EPA; thus, requiring additional controls for their use and disposal.

To eliminate many of these prior-art disadvantages in using harsh liquid chemicals for sanitizing and disinfecting objects found in high-use and high-risk environments, the method of using ozone to disinfect and sanitize target objects has seen a tremendous surge over the last few years and is expected to see continued exponential growth for many years to come. Ozone is a powerful oxidizing gas that can be formed from ambient air by use of electrical discharge or ultraviolet light, wherein ozone comprises 3 oxygen molecules (O₃). The first use of the word “ozone” was in 1840, in a writing by the German chemist Christian Friedrich Schonbein (1799-1868). Seventeen years later, in 1857 the first ozone generator was manufactured in Berlin, by the German inventor Ernst Werner von Siemens (1816-1892), wherein ozone was used in an attempt to destroy microorganisms in water. Then, in 1873, Cornelius Benjamin Fox (1839-1922) experimentally confirmed the ability of ozone to eliminate microorganisms. In 1896, Nikola Tesla (1856-1943) was issued U.S. Pat. No. 568,177 for an “Apparatus for Producing Ozone,” wherein ozone was formed by use of a corona discharge ozone generator having charged metal plates to act on ambient air. Subsequently, in 1900, Nikola Tesla founded the “Tesla Ozone Co.” to manufacture and sell ozone generators for medical use.

Following multiple laboratory results showing the safe and effective use of ozone as a disinfectant (at least a 3 log 10 or 99.9% reduction in virus titers) and a sanitizer (at least a 3 log 10 or 99.9% reduction in bacterial count), ozone has been used since the early 1900's to treat drinking water. In fact, almost all bottled water today is treated with ozone. Additionally, in 2000, the U.S. Department of Agriculture approved the use of ozone for meats and poultry, and in 2001, the U.S. Food and Drug Administration (FDA) approved the use of ozone for other foods. The World Health Organization (WHO) also recognizes ozone, when used properly, as an effective sanitizer for bacteria such as Staphylococcus aureus (including MSRA), Salmonella enterica, Escherichia coli (E-coli), Streptococcus pyogenes, Shigella dysenteriae, and Pseudomonas aeruginosa, and as an effective disinfectant for viruses such as respiratory syncytial virus (RSV), norovirus, and influenza. Although more studies are necessary, proper exposure to ozone has also shown promising results on non-porous and semi-porous surfaces as against viruses from the coronavirus family including SARS-COV-2, which causes Covid-19.

Ozone in lower concentrations (i.e., less than 0.05 ppm) is considered safe for indoor use by OSHA and EPA. However, to achieve the 3 log₁₀ reduction in virus titers and bacterial count, ozone must be at sustained levels much higher than 0.05 ppm; and, as such, for the purpose of disinfecting and sanitizing objects used in high-use environments, sealed ozone treatment chambers are typically utilized. Generally speaking, target objects to be sanitized and disinfected are placed within the sealed ozone treatment chamber, wherein an ozone generator is used to raise the ozone level within the chamber for a specified period of time and then, only after the ozone levels have been reduced to safe levels, the chamber is opened and the target objects are removed for immediate reuse.

The use of ozone treatment chambers has a multitude of advantages in comparison to the method of using harsh liquid chemicals. One of the most beneficial advantages is that proper ozone treatment has proven to be more effective in the reduction of many viruses and bacteria as compared to using liquid chemicals. Additionally, because ozone is a gas, it is able to reach and treat cracks and crevices that liquid chemicals may not reach. Because ozone treatment chambers generate ozone locally from ambient air, there is no need to transport or store dangerous liquid chemicals. Additionally, during the ozone treatment process, attention from personnel is not required, and, after use, target objects are immediately ready to be reused with no wiping or drying necessary; thus, labor costs are substantially reduced. Ozone treatment chambers also do not require or generate waste product that must be properly handled and disposed of, such as wipes, towels, containers, and gloves. Unlike ozone treatment chambers, harsh liquid chemicals often leave residue and an unpleasant odor. Ozone treatment chambers, like the present invention, are also energy and resource efficient, wherein less than 1 oz of water is used and less than 3 cents cost of electricity is used per treatment cycle.

As a result of the recent Covid-19 pandemic, which to date has been attributed to approximately 7 million deaths, as many as 1 billion infections and a total societal cost of more than 100 trillion dollars, coupled with the millions of deaths and billions of dollars spent annually on other viral and bacterial infections, the use of ozone treatment chambers for sanitizing and disinfecting objects in high-use environments has seen tremendous growth and innovation. Consequently, ozone treatment chambers have become a necessary and effective tool for minimizing the enormous societal cost resulting from the exposure and resultant illnesses caused by a plurality of bacteria and viruses carried on everyday objects. As such, even minor improvements in this field can have a significant positive impact on both the overall health and financial welfare of society.

Although there have been many attempts to design efficient and effective ozone treatment chambers, there are many deficiencies in these designs. For instance, as studies have shown, the use of humidity, at optimal percentages and optimal duration (as discussed further below), have a significant positive impact on the effectiveness of ozone treatment by the enabling the production of hydroxyl radicals. Hydroxyl radicals (OH) are a highly reactive product of ozone decomposition. Managing ozone level and humidity is critical in the production of hydroxyl radicals. The hydroxyl radical (OH) is the major intermediate reactive responsible for organic substrate oxidation. Hydroxyl radicals are extremely reactive and react as soon as they are formed. Their lifetime is less than a second, and they damage the majority of organic compounds, including bacteria and virus. Ozone and water must be continuously supplied at their optimum levels to generate hydroxyl radicals to the required levels for disinfection and sanitization.

However, some prior designs fail to incorporate the use of humidity and/or fail to provide a sufficient duration and combination to adequately disinfect and sanitize. In addition to the duration of treatment, it is also critical that the level of ozone in terms of parts per million (ppm) be high enough to effectuate the desired result. Failure to meet either these requirements (ppm or duration of treatment) could result in inadequate reduction (99.9%) of the applicable viruses and bacteria. Even though there have been attempts in prior designs to incorporate humidity into their treatment process, they fail to properly manage the humidity levels, wherein, for example, the objects to be sanitized/disinfected already have a high level of moisture content such as used sporting equipment, uniforms, personal protective equipment (PPE), and/or the like. Moreover, many prior designs also fail to remove moisture through a heating phase as may be needed for these types of items. Additionally, prior ozone treatment chamber designs fail to adequately couple a managed ozone treatment with a sufficient heat phase to effectively eradicate bed bugs, lice, and their eggs. It should also be noted that prior designs also fail to provide an easy means for the user to add additional water and/or to repair or replace the moisture generating means.

Another deficiency seen in prior attempts is their inability or inefficiency in converting ozone back to oxygen. More specifically, the high levels of ozone needed to adequately disinfect and sanitize is harmful to humans and animals. Consequently, to reduce this risk, additional time must be added to the treatment process to allow ozone to naturally decay to oxygen and/or use an ozone destructor to speed up this process. To facilitate same, various sensors must be incorporated to ensure that the level of ozone inside the chamber is at the optimal level during use and that, prior to opening the chamber, the level of ozone has been reduced to safe levels. Some prior attempts fail to provide adequate ozone destruction; thus, increasing the amount of time needed prior to the opening of the ozone treatment chamber. Additionally, some prior attempts fail to adequately detect ozone levels outside of the chamber; thereby, potentially failing to discover potential leaks. As another deficiency, unlike the present invention, some prior designs also fail to seal the inside of the chamber to further reduce the risk of high-level ozone exposure.

Consequently, as can be seen, there is a need for an apparatus, system, and method for disinfecting and sanitizing objects used in high-use environments that delivers an adequate means for providing and managing ozone exposure, humidity levels, ozone destruction, and heat cycles in a safe and efficient manner to provide adequate disinfection and sanitization of various viruses and bacteria, respectively. Accordingly, it is to the disclosure of such an apparatus, system, and method that the following is directed.

### SUMMARY

The subject matter of the present disclosure relates, in various embodiments, to an apparatus, system, and method that solves the above-discussed deficiencies by providing a new and improved ozone treatment chamber that effectively and efficiently disinfects and sanitizes a multitude of target objects.

According to some embodiments, the present invention broadly comprises a housing unit, an ozone generator, a humidity generator, an ozone destructor, and a control system for managing same. In alternate embodiments, in addition to the above structures, a heating system is provided to assist with drying if needed and/or to eradicate lice, bed bugs, and their eggs.

A feature and benefit of the present invention is its ability to disinfectant (at least a 3 log₁₀ or 99.9% reduction in virus titers) and sanitize (at least a 3 log 10 or 99.9% reduction in bacterial count) a plurality of viruses and bacteria, respectively; thus, eliminating the need for harsh chemical liquids. Consequently, less waste such as wipes, towels, gloves, and containers are generated.

Another feature and benefit of the present invention is its ability to locally generate ozone gas from ambient air at an extremely low energy consumption per treatment.

Another feature and benefit of the present invention is its ability to generate humidity in combination with ozone gas to allow for a more thorough and efficient means of disinfecting and sanitizing objects that could potentially carry viruses and bacteria.

Another feature and benefit of the present invention is the use of a piezo mister to add humidity to an ozone treatment, coupled with a hinged cage to facilitate the ease of adding water and/or replacing the piezo mister without the need for tools.

Another feature and benefit of the present invention is its ability to rapidly convert ozone back to oxygen via a catalyst; thereby, reducing the cycle time needed to reach safe levels of ozone.

Another feature and benefit of the present invention is a control system that utilizes multiple sensors and safety features to ensure safe use. More specifically, the following is included in a preferred embodiment: a sweep button to ensure the inside chamber is clear of humans or other animals prior to starting; a magnetic lock that ensures the chamber cannot be opened until safe ozone levels have been reached; internal sensors to ensure the proper level of ozone and humidity are present during treatment and that after treatment, safe levels of ozone levels are achieved prior to allowing user to open the chamber; external sensors to detect possibility of ozone leakage and exterior level of humidity; and viewing windows/ports to further confirm that no human or other animal are inside during treatment.

Another feature and benefit of the present invention is the use of rolling trays or carts that allow a multitude of objects to be simultaneously sanitized and disinfected; thus, substantially reducing labor intensive operations as required with liquid cleaners such as spraying, hand wiping, and drying of each individual object.

Another feature and benefit of the present invention is an easily refillable water tray; thus, eliminating the need to connect the present invention to a water source/line; and, thereby, allowing the present invention to be truly mobile and usable in a multitude of applications and environments.

Another feature and benefit of an alternate embodiment of the present invention is the use of one or more mesh filters to facilitate the evaporation of water, coupled with the ease of changing the filters, without the need for tools.

Another feature and benefit of some alternate embodiments of the present invention is the addition of a heating phase, wherein objects having high moisture content such as sporting equipment, uniforms, PPE, and/or the like may be dried to further ensure safe use. Additionally, the heating phase can also be used in combination with ozone treatment to eradicate lice, bed bugs, and their eggs.

These, and other features, advantages, and benefits shown by the various embodiments of the present subject matter, and related processes for creating them, as set forth within the present disclosure, will become more apparent to those of ordinary skill in the art after review of the following Detailed Description of Illustrative Embodiments and Claims in light of the accompanying drawing Figures.

### BRIEF DESCRIPTION OF THE DRAWINGS

Accordingly, the within disclosure will be best understood through consideration of, and with reference to, the following drawing Figures, viewed in conjunction with the Detailed Description of Illustrative Embodiments referring thereto, in which like reference numbers throughout the various Figures designate like structure, and in which:

FIG. 1A depicts a front view of the preferred embodiment of the present invention, shown in a closed position, in accordance with the subject matter of the present disclosure;

FIG. 1B depicts a front view of the preferred embodiment of the present invention, shown in an open position, in accordance with the subject matter of the present disclosure;

FIG. 1C depicts a top perspective view of the preferred embodiment of the present invention shown in a closed position, in accordance with the subject matter of the present disclosure;

FIG. 1D depicts a perspective view of the preferred embodiment of the present invention shown in an open position wherein the doors have been removed, in accordance with the subject matter of the present disclosure;

FIGS. 2A-2C, inclusive, depict views of the ozone generating system of the preferred embodiment of the present invention in accordance with the subject matter of the present disclosure;

FIGS. 3A-3B, inclusive, depict views of the humidity generating system of a preferred embodiment of the present invention, in accordance with the subject matter of the present disclosure;

FIGS. 3C-3E, inclusive, depict perspective views of the humidity generating system of a preferred embodiment of the present invention, in accordance with the subject matter of the present disclosure;

FIG. 3F depicts a perspective view of the humidity generating system of a preferred embodiment of the present invention, in accordance with the subject matter of the present disclosure;

FIGS. 4A-4D, inclusive, depict perspective views of the humidity generating system of an alternate embodiment of the present invention, in accordance with the subject matter of the present disclosure;

FIG. 4E depicts a perspective view of the humidity generating system of an alternate embodiment of the present invention, in accordance with the subject matter of the present disclosure;

FIGS. 5A-5E, inclusive, depict views of the ozone destruction system of a preferred embodiment of the present invention, in accordance with the subject matter of the present disclosure;

FIG. 6 depicts a view of part of the control system of a preferred embodiment of the present invention, in accordance with the subject matter of the present disclosure;

FIG. 7A depicts a view of a sensor of a control system of a preferred embodiment of the present invention, in accordance with the subject matter of the present disclosure;

FIG. 7B depicts a view of the sensor of the control system of a preferred embodiment of the present invention, in accordance with the subject matter of the present disclosure;

FIG. 8 depicts a view of the PLC wiring connections for the control system of a preferred embodiment of the present invention, in accordance with the subject matter of the present disclosure;

FIG. 9 depicts a view of the expansion module one wiring connections for the control system of a preferred embodiment of the present invention, in accordance with the subject matter of the present disclosure;

FIGS. 10A-10B, inclusive, depict front views of an alternate embodiment of the present invention showing in the closed position and in the open position, in accordance with the subject matter of the present disclosure; and

FIG. 11 depicts a view of a heating system of a preferred embodiment of the present invention, in accordance with the subject matter of the present disclosure.

It is to be noted that the drawing Figures presented are intended solely for the purpose of illustration and that they are, therefore, neither desired nor intended to limit the inventive subject matter to any or all of the exact details of construction shown, except insofar as they may be deemed essential to the claimed invention.

### DETAILED DESCRIPTION OF ILLUSTRATIVE EMBODIMENTS

In describing the several embodiments illustrated in the Figures, specific terminology is employed for the sake of clarity. The inventive subject matter, however, is not intended to be limited to the specific terminology so selected, and it is to be understood that each specific element includes all technical equivalents that operate in a similar manner to accomplish a similar purpose. Additionally, in the Figures, like reference numerals and like description shall be used to designate corresponding elements, parts, and functionality throughout the several Figures.

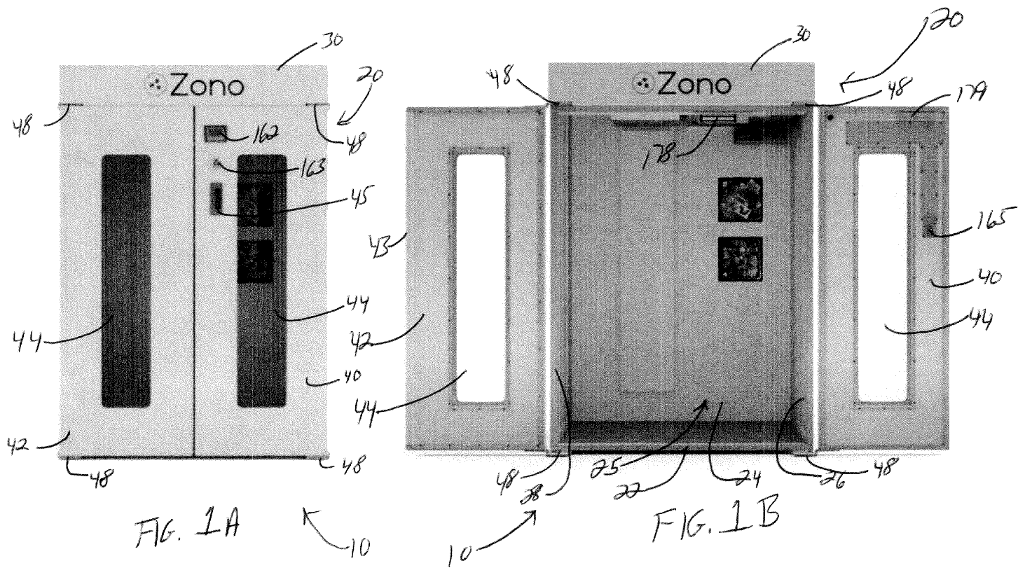

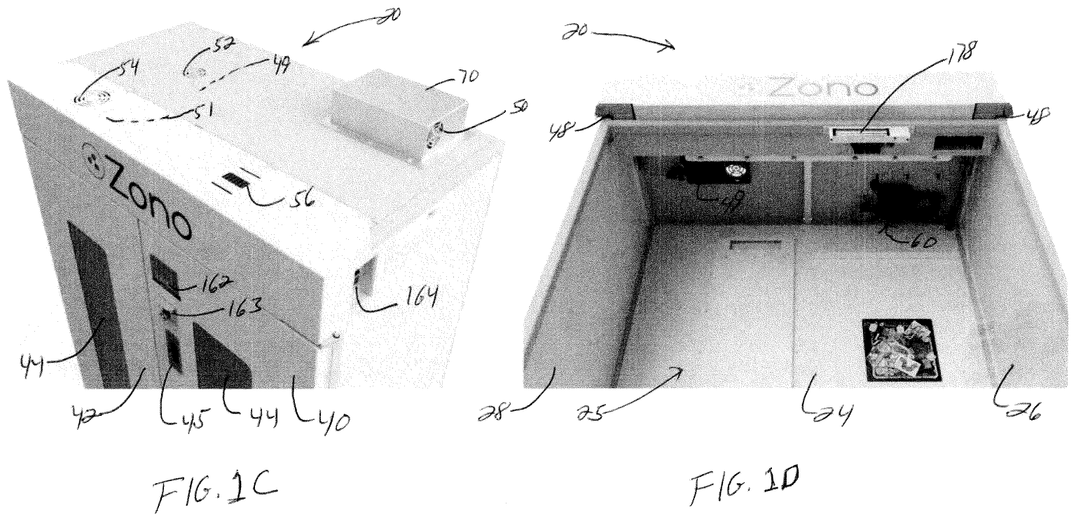

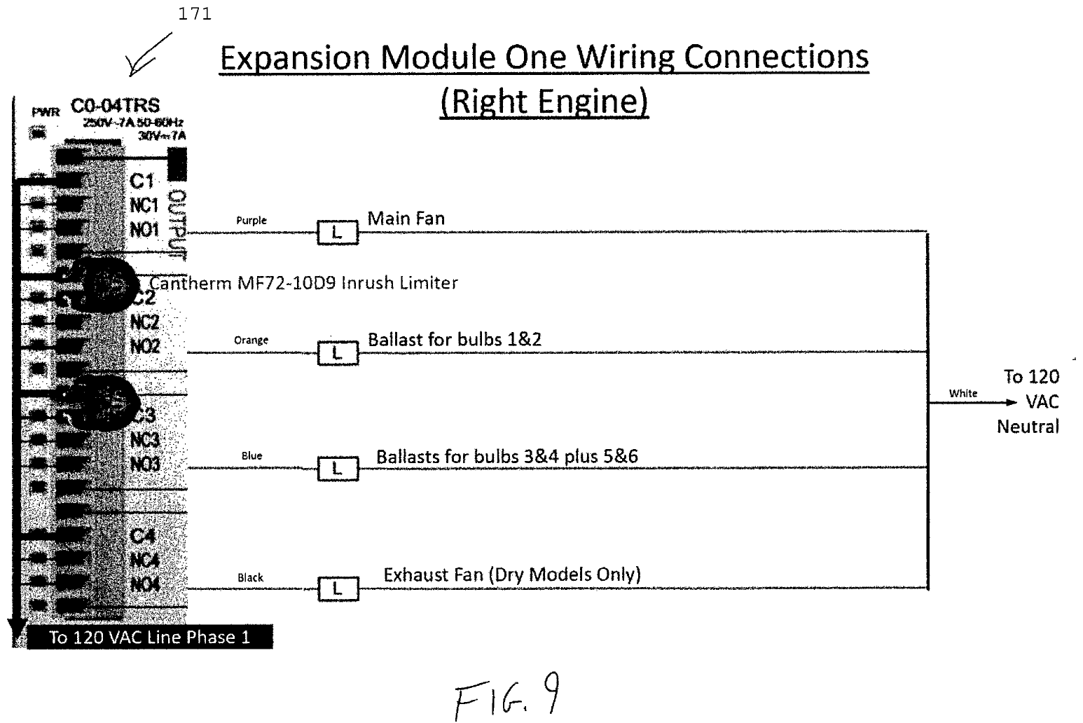

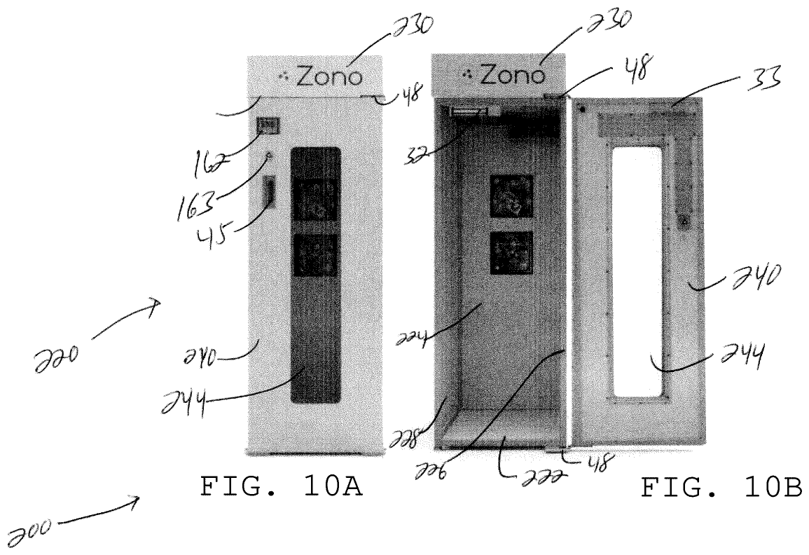

Turning now to the FIGS. 1-10 generally, and more specifically to FIGS. 1A-1D, inclusive, the preferred embodiment of the present invention 10, comprises a housing unit 20, an ozone generation system 60, a humidity generation system 80, an ozone destruction system 120, and a control system 160. In alternate embodiments, the present invention 10 further comprises a heating system 250.

Housing unit 20 is generally rectangular shaped and comprises a floor member 22, a rear wall 24, a first side wall 26, a second side wall 28, an upper member 30, a first door 40, and a second door 42, wherein said members are connected to define a closeable chamber 25 therebetween. Chamber 25 is a treatment area in which target items (not shown) are placed to be disinfected/sanitized/heated. Housing unit 20 has attached thereon ozone generation system 60, a humidity generation system 80, an ozone destruction system 120, and a control system 160, wherein in a preferred embodiment, said systems are carried by upper member 30 of housing unit 20.

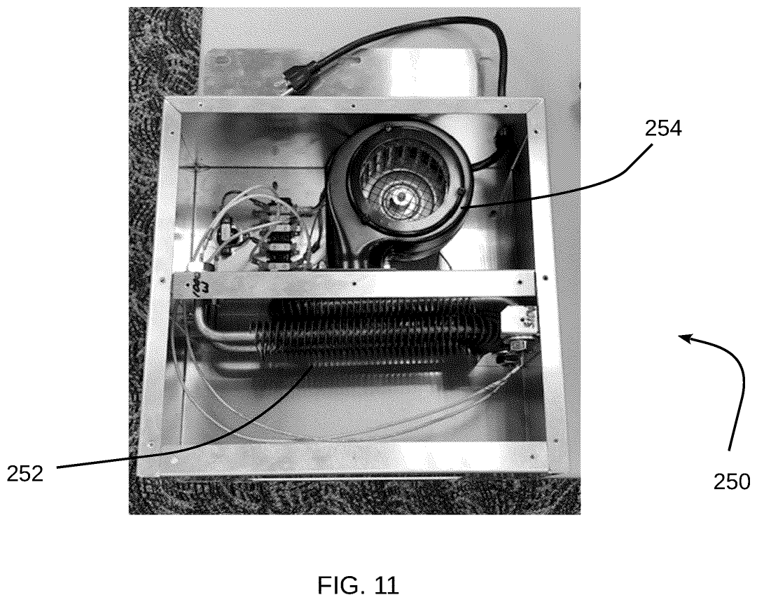

Additionally, in embodiments utilizing a heating system 250, housing unit 20 also carries said heating system 250. Best seen with reference to FIG. 11, heating system 250 preferably utilizes 120 volt, 1440 watt resistance heating elements 252, in association with circulating blower 254. Heating system 250 typically mounts to the left of the engine, above the plenum, in a 2-door embodiment, and in the bottom of a cart for a single door machine embodiment. In a 2-door embodiment, there is a rectangular hole in the rear of heating system 250 that directs and feeds air into the plenum. There is no need for a plenum-mounted heating system in a single door embodiment, because warm air rises and does not require further forced circulation. It is further noted, operationally, that it is undesirable to run the heater during an ozone cycle, because temperature changes impact the inside ozone sensor readings. This preference is enabled via the control system 160 discussed further hereinbelow.

It is contemplated in alternate embodiments that said systems could also be attached to and carried by floor member 22, rear wall 24, first side wall 26, and/or second side wall 28. First door 40 and second door 42 are attached to upper member 30 and floor member 22 via hinges 48, wherein first door 40 and second door 42 are dimensioned to seal chamber 25 when closed. In the preferred embodiment, the sealing of first door 40 and second door 42 is via a mechanical fit utilizing one or more PVC magnetic gasket affixed around the perimeter of each door; however, in alternate embodiments it is contemplated that other known sealing means may be utilized. In this way, the chamber is essentially air tight. A lip 43 extends from second door 42 such that second door 42 is closed first and then first door 40 is closed to rest over lip 43, wherein when locked, second door 42 cannot be opened without first opening first door 40.

As will be described in more detail below in the discussion of control system 160, a magnetic lock 178 and an armature 179 are provided to secure first door 40 and second door 42 in a closed and locked position during a treatment cycle and until control system 160 determines that ozone levels are at a safe level for the user to open same. First door 40 and second door 42 each further comprise a viewing window 44 to allow a user to see within chamber 25 during operation to further ensure that no human or other animal is therein. Viewing window 44 preferably extends a substantial length of first door 40 and second door 42 to assist a user in visual inspection of the entire chamber 25 area prior to use.

As will also be discussed in further detail below, an interior emergency stop button 165 is attached to the interior of first door 40 and in electronic communication with control system 160; wherein, in the unlikely event a person is inside chamber 25, said person can push said interior emergency stop button 165 to immediate shut down operation and unlock magnetic lock 178 to allow first door 40 to be opened. Similarly, an exterior emergency stop button 163 is attached to the exterior of first door 40 and in electronic communication with control system 160, wherein a user can push said exterior emergency stop button 163 to immediate shut down operation and unlock magnetic lock 178 to allow first door 40 to be opened.

Loss of power will disable the magnetic door lock. As well, depressing an emergency stop button will unlock the door(s), regardless of ozone concentration, and will run the destructor for a predetermined minimum of 10 minutes. If power is lost during a cycle, then the doors will unlock; however, ozone decay will only occur naturally and through incidental exposure to the catalyst.

Attached to the exterior of first door 40 is handle 45 to allow a user to open same upon completion of a cycle or to load target items. In preferred embodiments, if the doors are left unlocked for more than 10 minutes while the machine is turned on, then a warning buzzer will chime until doors are locked or until the machine is turned off.

Also attached to the exterior of first door 40 is control panel 162 of control system 160, wherein control panel 162 will be discussed further below. Although in the preferred embodiment armature plate 179, control panel 162, handle 45, interior emergency stop button 165, and exterior emergency stop button 163 are carried by first door 40, it is contemplated that some or all of said items could be carried by second door 42. In the event that magnetic lock 178 and armature 179 are relocated to second door 42, one skilled in the art would understand that lip 43 would need to extend from first door 40.

Now referring more specifically to FIG. 1C, a top perspective view of housing unit 20 is shown with first ventilated port 50, second ventilated port 52, third ventilated port 54, exterior sensor port 56, and engine encasement 70, wherein engine encasement 70 comprises ozone generation system 60, ozone destruction system 120, and main fan 62. Ventilated port 50 is formed within engine encasement 70 to allow ambient air to enter chamber 25 and circulate over UV lamps 64 via the operation of main fan 62. Ventilated port 52 and third ventilated port 54 are to allow ambient air inside chamber 25 and air inside chamber 25 to be exhausted via first inlet fan 49 and second exhaust fan 51, respectively, after a heat cycle or dry cycle, as more fully described below. Coupled with each ventilated port 52, 54 is first vent valve 53 and second vent valve 55, respectively, wherein vent valves 53, 55 are in electrical communication with PLC 170 of control system 160. Vent valves 53, 55 are closed except during a drying phase as fully described below.

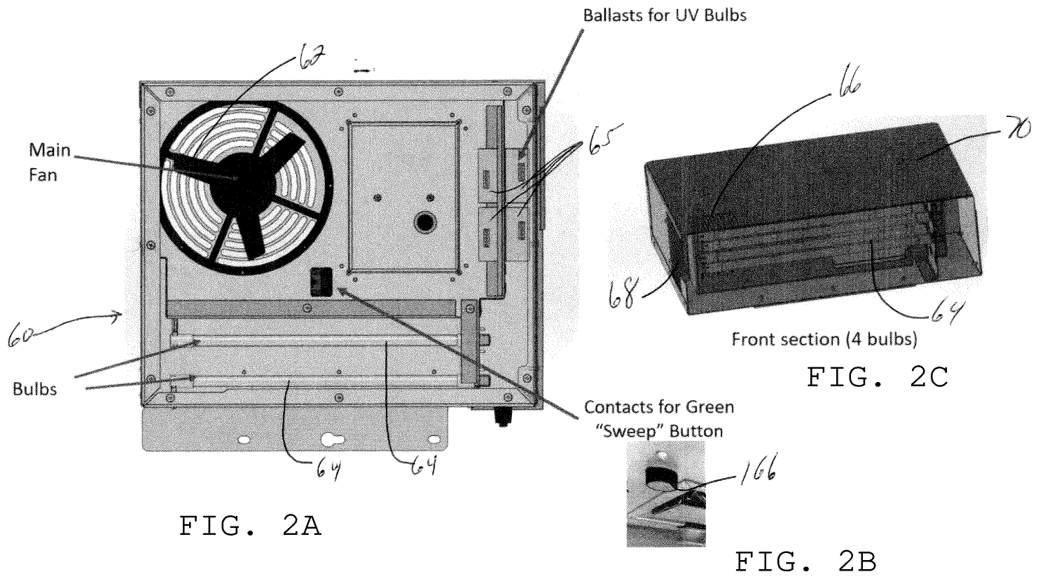

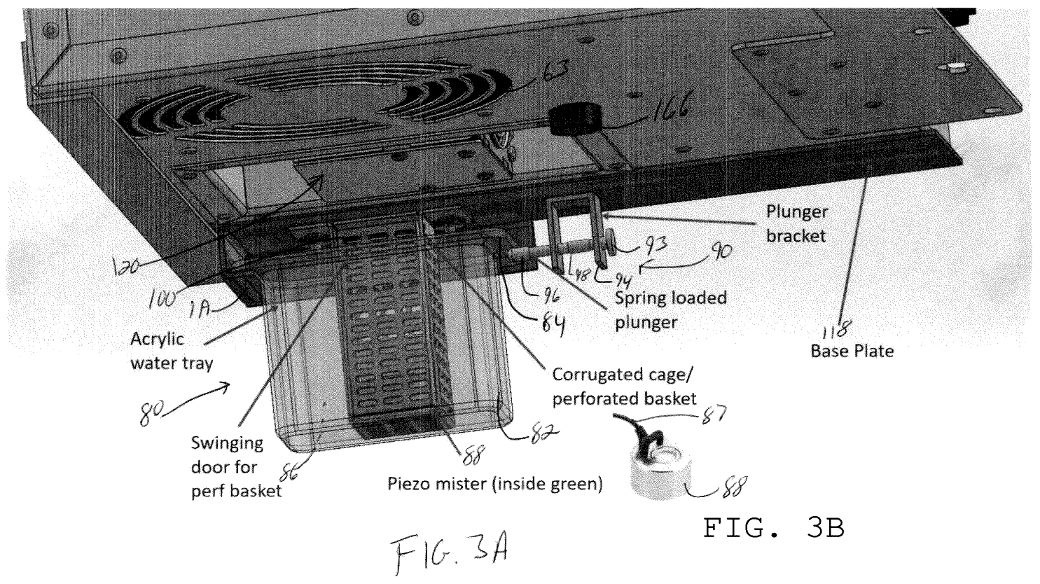

Now, with reference to FIGS. 2A-2C, inclusive, ozone generation system 60 comprises main fan 62, UV lamps 64, ballasts 65 and frame 71, wherein frame 71 contains preferably four (4) UV lamps 64 and four (4) ballasts 65, and wherein UV lamps 64 operate between a wave length of 160-240 nm, and preferably at a wave length of 185 nm; thus, allowing the lamps to produce ozone from ambient air. UV lamps 64 require a specific range of voltage and current to operate correctly, therefore each ballast 65 is electrically connected to each UV lamp 64 to regulate the flow of electricity to each UV lamp 64 to ensure that each receives the correct amount of power. In the preferred embodiment, each UV lamp 64 has a power rating of 48 watts. UV lamps 64 are attached to and carried by frame 71, wherein UV lamps 64 are electrically connected to control system 160. Frame 71 is positioned in close proximity to main fan 62, wherein first vents 66 and second vents 68 in frame 71 allow air flow to traverse over UV lamps 64 to facilitate the generation of ozone. Although the preferred embodiment utilizes four (4) UV lamps 64 having a power rating of 48 watts, it is contemplated in alternate embodiment that six (6) or more UV lamps and/or UV lamps 64 with differing power ratings may be utilized based on known parameters, such as the volume of chamber 25 and the desired ozone level for treatment. Likewise, with smaller embodiments having a chamber 25 of smaller volume, it is contemplated that fewer UV lamps 64 may be utilized, as long as the desired level of ozone treatment (20 ppm/20,000 ppb) is achieved and sustained within a reasonable treatment period (preferably approximately 20 minutes) to achieve 99.9% reduction in viral titers and a 99.9% reduction in bacteria count. Additionally, although the preferred method to create ozone is via said UV lamps 64, it is contemplated that other known methods such corona discharge could be utilized in alternate embodiments.

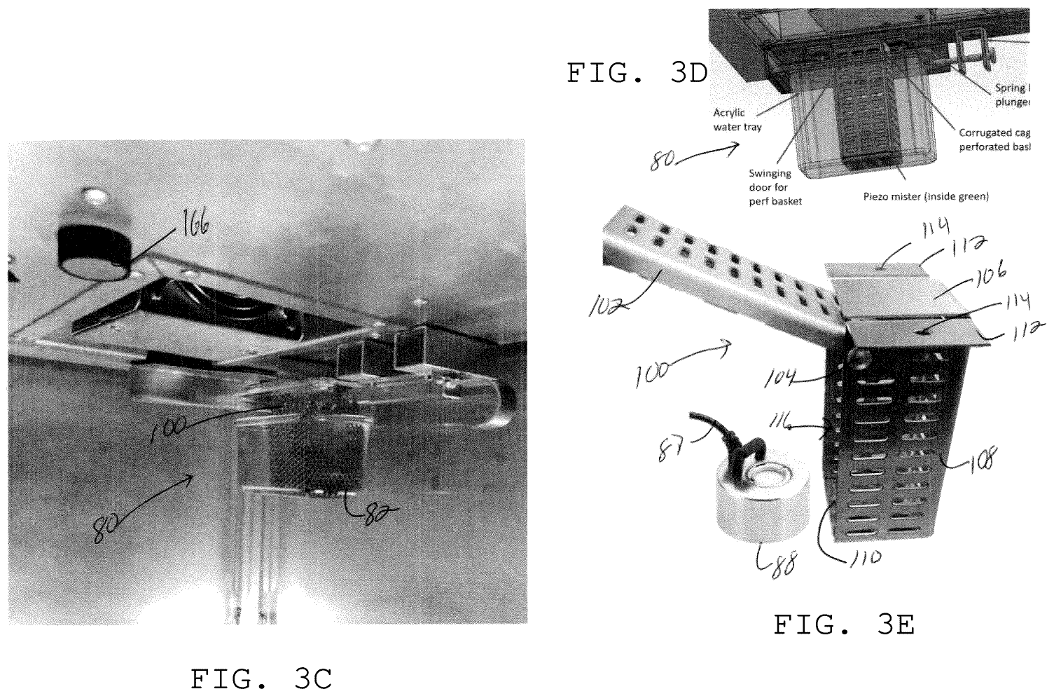

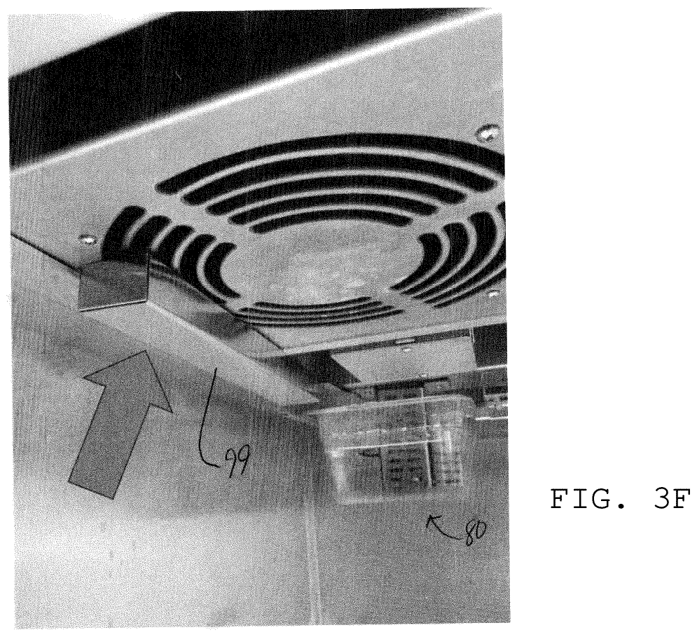

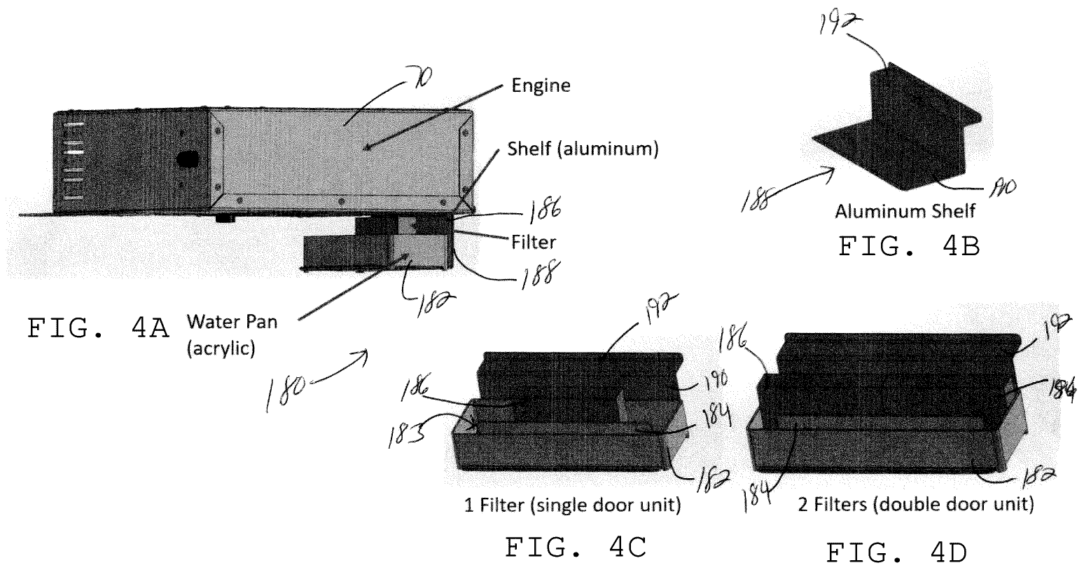

Now, with reference to FIGS. 3A-FIG. 3F, inclusive, humidity generation system 80, preferably comprises water tray 82, Piezo mister 88, plunger mechanism 90, and perforated basket 100 attached to and carried by base plate 118, wherein base plate 118 comprises a ledge 119, and wherein base plate 118 is attached to engine encasement 70. In alternate embodiments, it is contemplated that base plate 118 could be attached to any member of housing 20, as long as airflow from main fan 62 is directed over humidity generation system 80. Water tray 82 is a one-sided open generally cuboid shape having an inside volume 86 for receiving distilled water. It is contemplated that water tray 82 could have any shape as long as an inside volume 86 is defined therein to hold distilled water for the generation of humidity.

Additionally, although distilled water is the preferred liquid for humidification, it is contemplated that other liquids such as spring water, filtered water, or tap water with or without additives may be utilized; however, some waters, especially tap water, typically contain minerals that will leave deposits and scale thus requiring more frequent sanitizing and disinfecting, and the risk of added microorganisms; and, therefore, is not preferred.

Water tray 82 further comprises lip 84 around and extending outwardly from the open perimeter thereof for securing water tray 82 in position. More specifically, in the secured/use position, lip 84 of water tray 82 rests partially upon ledge 119 of base plate 118. To facilitate the easy removal of water tray 82 for refilling and/or cleaning, a plunge mechanism 90 is utilized under one edge of lip 84, wherein plunge mechanism comprises spring-loaded plunger 92 and bracket 94 having a through-hole 97 therethrough. Spring-loaded plunger 92 comprises a head portion 93, distal end 96, and intermediate area 98, wherein spring-loaded plunger extends through through-hole 97 of bracket 94, and wherein distal end 96 rests under a portion of lip 84 of water tray 82. In the resting position, spring-loaded plunger 92 secures water tray 82 in position.

When water needs to be added to water tray 82, the user pulls the head portion 93 of spring-loaded plunger 92, thereby releasing distal end 92 from lip 84, allowing water tray 82 to be easily removed. Further, attached to and carried by base plate 118 is corrugated cage 100, wherein corrugated cage 100 is defined by swinging door 102, 3-sided wall member 108, top member 106, bottom member 110, shoulders 112, and through-holes 114, wherein an inside area 116 is formed therebetween. Shoulders 112 having through-holes 114 allow corrugated cage 100 to be attached to base plate 118 of housing 10.

In use, known Piezo mister 88 is placed within inside area 116 on bottom member 110. In order to allow a user to inspect and/or replace Piezo mister 88, swinging door 102 is attached to 3-sided wall member 108 at hinged joints 104. Corrugated cage 100 is dimensioned to fit within and generally at the center of inside area 86 of water tray 82. Once Piezo mister 88 is placed on bottom member 110 of corrugated cage 100, swinging door 102 is closed and water tray 82, having water therein, is placed in position, by first pulling spring-loaded plunger 92 and placing lip 84 on ledge 119, wherein corrugated cage 100 rests inside of water tray 82, and wherein Piezo mister 88 is submerged in the water.

Piezo misters 88 have a known optimum operating water level or depth requirement of approximately 1 inch above the piezo disk, wherein the level of water inside water tray 82 is, preferably at or near this optimum operating water level or depth. To facilitate the filling of water to this optimum operating depth, a “fill line” (not shown) is provide on water tray 82 for the user.

Piezo mister 88 is electrically connected to control system 160 via wire 87. After its determined that the level of humidity inside chamber 25 is below the minimum start point humidity level (as described below), PLC 170 of control system 160 will send electrical current to the atomizer of Piezo mister 88, wherein rapid ultrasonic frequencies are generated thus causing the water to be pushed through the mesh disc producing tiny droplets or mist. The Piezo mister 88 is placed in close proximity to main fan 62 and/or an air duct 99 (as shown in FIG. 3F) is added such that a portion of air from main fan 62 is directed to humidity generation system 80 to facilitate the humidification process in chamber 25. In the preferred embodiment, air duct 99 is utilized for this process; however, in alternate embodiments, it is contemplated that air duct 99 is not utilized. It should be noted that in alternate embodiments, it is contemplated that more than one Piezo mister 88 may be utilized, wherein the speed at which humidification takes place is increased.

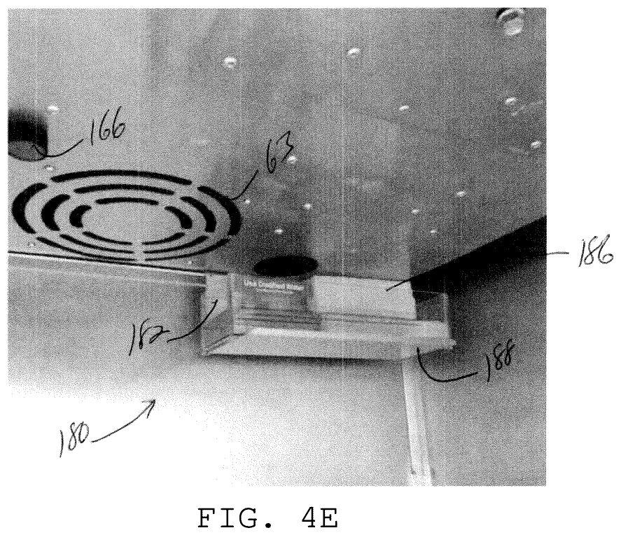

Now, with reference to FIGS. 4A-4E, inclusive, an alternate embodiment of a humidity generation system 180 is shown, wherein humidity generation system 180 comprises water pan 182, shelf 188, and wicking filters 186. Water pan 182 is generally a rectangular-shaped pan having a fill area 183 and a divider 184, wherein divider 184 is generally positioned longitudinally within fill area 183 of water pan 182. Water pan 182 is dimensioned to partially receive two (2) each of wicking filters 186 on each side of divider 184, such that, in this alternate embodiment, water pan 182 could hold a total of four (4) wicking filters 186.

In a single door alternate embodiment 200 as shown in FIGS. 10A-10B, inclusive, only one (1) wicking filter 186 is needed, whereas in the 2-door preferred embodiment 10, two (2) wicking filters 186 is utilized. However, it is contemplated in alternate embodiments that more wicking filters 186 may be utilized to increase the speed at which humidification takes place.

Only the lower portion of wicking filters 186 are submerged in water such that as air passes over wicking filters 186, water travels up and then evaporates to increase the moisture, and thus, humidity within chamber 25. Water pan 182 is held in position proximal to main fan 82 via shelf 188, wherein shelf 188 is generally L-shaped having a ledge 192 and a horizontal member 190, and wherein ledge 192 is used to attach shelf 188 to base plate 118, and wherein horizontal member 190 is dimensioned to rest thereon water pan 182.

In use, water pan 182 is filled with clean water, preferably is distilled, and wicking filters 186 are placed therein. Then, water pan 182 is placed on horizontal member 190 of shelf 188. Similarly to the preferred embodiment, it is contemplated that air duct 99 could be utilized in alternate embodiments to facilitate air flow from the main fan to humidity generation system 180.

In use, before the present invention can be safely opened after a treatment, ozone levels within chamber 25, as measured by internal sensor 172, must be below OSHA safe level requirements of 0.10 ppm. In the present invention, acceptable ozone levels are set to a level of less than 200 ppb before control 160 will allow the user to open doors 40, 42. Although ozone, over time, will naturally decay to oxygen (half-life at typical room temperature of about 7-10 minutes), this period of time to allow the ozone to go from the preferred treatment level of 20 ppm/20,000 ppb to safe levels below 0.10 ppm could take well over an hour.

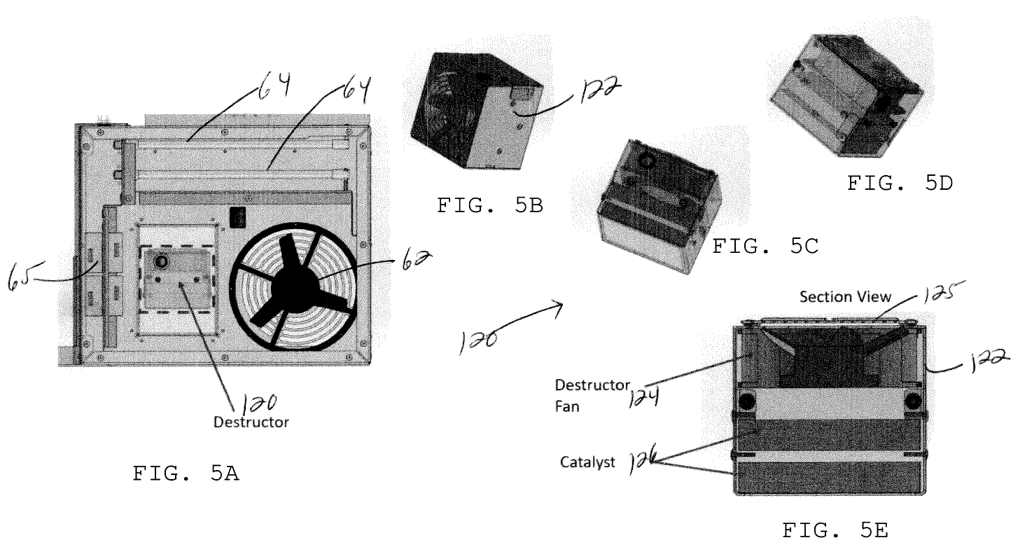

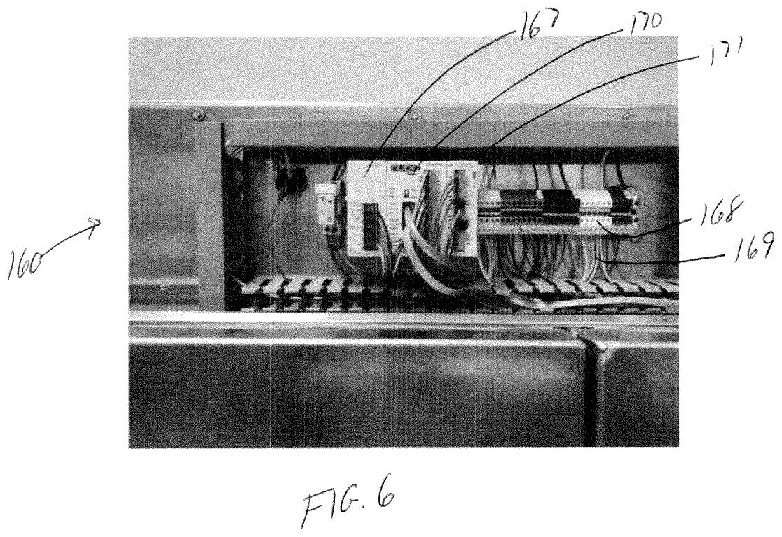

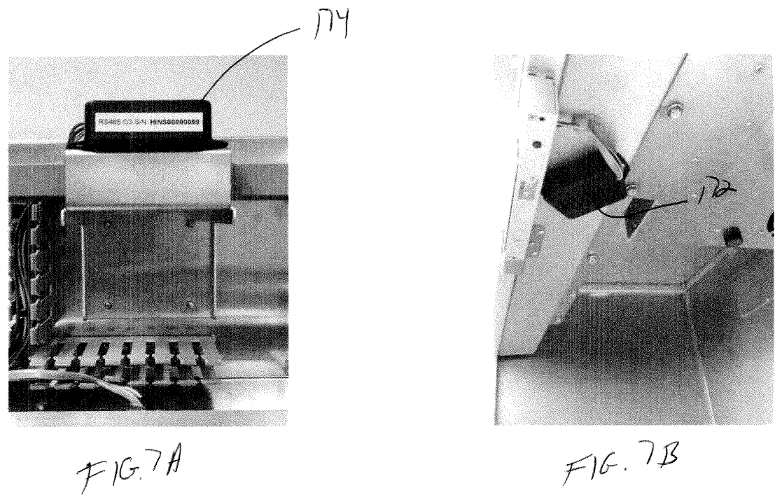

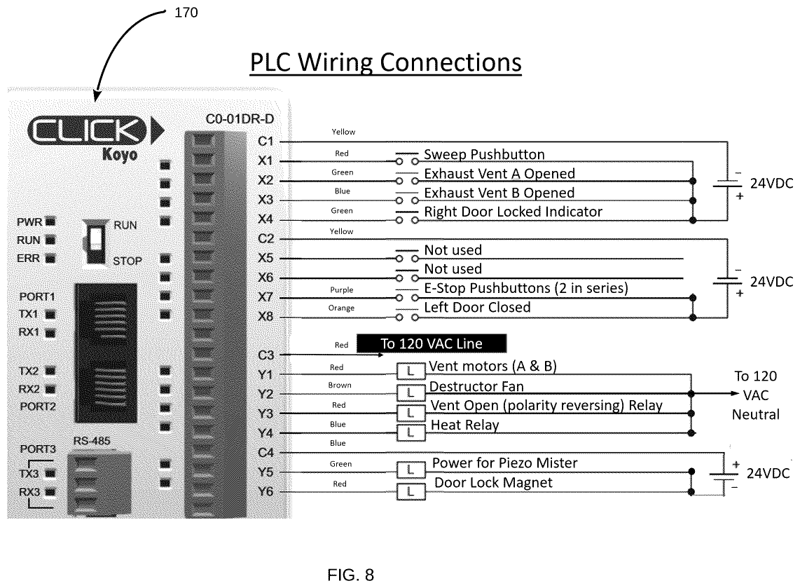

As such, to expedite the destruction of ozone and thus speed up the entire cycle time, an ozone destruction system 120 is utilized. With reference to FIGS. 5A-5E, inclusive, ozone destruction system 120 is attached within engine encasement 70 and comprises case 122, destructor fan 124, and catalyst 126, wherein destructor fan 124 and catalyst 126 are attached to and carried by case 122. Case 122 has an open inlet 125 and an open outlet 127, wherein destructor fan 124 is positioned proximal to inlet 125 and wherein catalyst 126 is positioned downstream between destructor fan 124 and outlet 127, such that when destructor fan 124 is activated by control system 160 after an ozone treatment, ozonated air within chamber 25 is allowed to flow into inlet 125, over catalyst 126 and out of outlet 127. Catalyst 126 serves to expedite the chemical reaction of ozone (O₃) to oxygen (O₂). In the preferred embodiment, catalyst 126 is manganese dioxide (MnO₂); however, other known catalyst such as oxides of Co, Cu, Fe, Ni, Si, Ti, Zr, Ag, and Al, or combination thereof, may be utilized. Catalyst 126, in the preferred embodiment, comprises two layers spaced apart approximately one fourth of an inch (¼″). This ¼″ air gap further facilitates the conversion of ozone to oxygen. This is because the introduction of an air gap reduces flow speed of the fluid through the 2nd catalyst layer, and increases total exposure of the fluid to the catalyst material, increasing destructor efficiency. Internal sensor 172 operates continuously, at a preferred rate of once per second, and measures the ozone level within chamber 25. Until internal sensor 172 indicates to controller 160 that said ozone levels are below 200 ppb, destructor fan 124 will continue to operate.

With continuing reference to FIG. 1A-1D, inclusive, and with further reference to FIGS. 6-9, inclusive, control system 160 comprises programmable logic controller (PLC) 170, expansion module 171, wiring 169, wire bus 168, external sensor 174, internal sensor 172, power regulator 167, main power switch 164, control panel 162, exterior emergency stop button 163, interior emergency stop button 165, sweep button 166, magnetic lock 178, armature 179, and a power source, wherein all of said components of control system 160 are in electronic communication. In the preferred embodiment, the above components of control system 160 are hardwired together via wiring 169; however, it is contemplated in alternate embodiments that some or all of said components could be electronically connected via any known wireless means. PLC 170 serves as the programmable brains of control system 160, wherein input data from the various components will, based on programmed perimeters (as discussed elsewhere herein), determine the output controls of PLC 170. More specifically, with a power source supplied and main power switch 164 opened, power regulator 167 provides the necessary power to control system 160, ozone generation system 60, humidity generation system 80, ozone destruction system 120, and heating system 250 (if applicable). Via wiring 169, expansion module 171, and wire bus 168, the components of the various systems are able to provide input to and receive output from PLC 170.

Now referring to FIGS. 10A-10B, inclusive, alternate embodiment 200 is shown, wherein alternate embodiment 200 is a single-door unit, and wherein, similar to preferred embodiment 10, alternate embodiment 200 comprises all the same systems and operates in the same manner as preferred embodiment 10. The primary differences between alternate embodiment 200 and preferred embodiment 10, are that housing 220 of alternate embodiment 200 is smaller in size, only has one door and, if alternate humidity generation system 180 is utilized, only one (1) wicking filter 186 is used as previously discussed. Notwithstanding, wick count ultimately is determined based upon ambient humidity conditions of the unit's location. Additionally, if alternate embodiment 200 is being operated with a heat unit, then the heat unit typically will be placed on the bottom tray of the cart. Consequently, only housing 220 will be detailed. Housing 220 is generally rectangular shaped and comprises a floor member 222, a rear wall 224, a first side wall 226, a second side wall 228, an upper member 230, and a door 240, wherein said members are connected to define a closeable chamber 25 therebetween. Similar to preferred embodiment 10, door 240 comprises a viewing window 244 to allow a user to see within chamber 25 during use.

A multitude of safety features are incorporated into the present device 10 and its alternate embodiments to ensure a safe operation for both the user and the surrounding environment. Generally, and as will be further detailed below in the discussion of the process steps, sweep button 166 is positioned on the inside of housing unit 20, wherein a user, prior to beginning a treatment cycle, inspects chamber 25 to ensure no humans or other animals are therein and then must push sweep button 166 before the control system 160 will allow the user to continue. As an additional safety feature, a user must also enter a code on control panel 162 before continuing. It is noted that, once the user pushes the sweep button, the treatment cycle must be started within thirty (30) seconds, or the cycle will time-out and be discontinued.

Once the above two preliminary steps are completed, the user can then activate a treatment cycle via control panel 162. Immediately, control system 160 energizes magnetic lock 178 to ensure that first door 40 and second door 42 are secured closed before and during said treatment cycle. If for any reason doors 40, 42 are opened, control system 160 shuts down said treatment cycle. Additionally, in the unlikely event that the user neglected to see a person inside of chamber 25, interior emergency stop button 165 is provided, wherein when pushed, control system 160 immediately shuts down said treatment cycle and unlocks doors 40, 42. An external emergency stop button 163 is also provided that functions similar to shut down and unlock in the event the user pushed same during an emergency situation.

In addition to these safety features, sensors are incorporated to provide various data (ozone levels, humidity, and temperature) to PLC 170, wherein under certain conditions that may indicate an issue or potentially unsafe parameter, PLC 170 can take corrective measures to remediate or mitigate the potentially unsafe condition. More specifically, if internal sensor 172 detects that the ozone levels inside chamber 25 are higher than the acceptable levels during a treatment cycle, this information is communicated to PLC 170 and then PLC 170 shuts down ozone generating system 60. In the highly unlikely event that ozone generating system 60 fails to shut down, PLC 170 can completely shut down the entire system. If external sensor 174 detects ozone on the exterior of housing 20, potentially indicating that there is a leak, this information is communicated to PLC 170, wherein PLC 170 can immediately activate ozone destruction system 120 and/or shut down the treatment cycle. Additionally, prior to allowing the user to be able to open doors 40, 42, PLC 170 must receive data from internal sensor 172 that the ozone levels inside chamber 25 have reached safe levels.

In addition to the above safety features, control system 160 controls the entire treatment cycle to ensure that the present invention performs as expected, whether that is a 99.9% reduction in viral titers, a 99.9% reduction in bacteria count, the eradication of lice, bedbugs, and their eggs, and/or a sufficient drying of damp/wet target objects.

The following is a breakdown of the preferred treatment cycle for the various applications. As referenced below, although the preferred exterior humidity setpoint, the interior minimum starting humidity setpoint, the ozone setpoint, the ozone overshoot period, the ozone treatment period, the heat setpoint, the heat treatment period, the drying treatment period, and the various steps are designated below for the preferred embodiment, it is contemplated that these values and steps can vary depending on various known factors such as, for exemplary purposes only, the virus or bacteria to be targeted and the objects being treated, wherein based on the present disclosure coupled with known scientific research, one skilled in the art would know how to vary these parameters and steps to accomplish the intended goal. It should also be noted that based on the disclosure herein, one skilled in the art would be able to program PLC 170 to accomplish the following parameters, other parameters and/or other steps as contemplated by the various alternate embodiments and other treatment cycles hereof and as desired by the user; and thus, the present invention should not be limited in any way to the following steps, order of steps, and/or parameters:

### Sanitizing Cycle

(a.) User loads chamber 25 with items to be treated;
(b.) User verifies that no person or other animal is inside chamber 25 and presses sweep button 166;
(c.) User closes doors 40, 42 and enters an access code on control panel 162;
(d.) User selects “Start Sanitizing Cycle” on control panel 162;
(e.) Control system 160 verifies that doors 40, 42 are closed and locked via magnetic lock 178;
(f.) Control system 160 checks outside humidity level via external sensor 174;
(g.) If outside humidity level is above setpoint (55% in a preferred embodiment), cycle begins;
(h.) If outside humidity level is below setpoint, the cycle pauses for two minutes to accurately measure the humidity level inside the machine via internal sensor 172;
(i.) If the inside humidity level is below the minimum starting setpoint (55% in a preferred embodiment), the cycle countdown timer pauses until humidity generation system 80 raises the level above the minimum starting setpoint. During this pause, control panel 162 displays “Preparing to Sanitize;”
(j.) Once the inside humidity level is above the minimum starting setpoint, ozone lamps 64 of ozone generation system 60 turn on and a cycle countdown timer begins (preferably 20 minutes);
(k.) Ozone lamps 64 remain on until internal sensor 172 detects an ozone reading exceeding the ozone setpoint (20 ppm/20,000 ppb in a preferred embodiment) for a predetermined “overshoot” period (90 seconds in a preferred embodiment);
(l.) If the ozone level in chamber 25 drops below the setpoint, ozone lamps 64 turn on until ozone reading via internal sensor 172 exceeds the setpoint for the predetermined “overshoot” period;
(m.) The previous step repeats until the timer reaches the end of the ozone treatment period (20 minutes in a preferred embodiment);
(n.) After the ozone treatment period, control system 160 initiates the ozone destruction period by turning on destructor fan 124 of ozone destruction system 120 to blow air across the manganese dioxide catalyst 126 to convert ozone to oxygen;
(o.) Air is circulated through catalyst 126 of ozone destruction system 120 until the ozone level drops to the level that is safe to open the door (under 20 ppm/20,000 ppb) typically 18 minutes in the preferred embodiment;
(p.) If necessary, the 18-minute destruct period will automatically be extended to ensure that the ozone level drops to the level that is safe to open the door;
(q.) Control panel 162 then displays that the cycle is complete;
(r.) If control system 160 detects that key cycle parameters were not met, control panel 162 notifies that user of the deficiency; and
(s.) The user can then enter an access code on control panel 162 to unlock doors 40, 42 and remove the treated items from chamber 25.

### Heat Cycle

(a.) User loads chamber 25 with items in to be treated;
(b.) User verifies that no person or other animal is inside chamber 25 and presses sweep button 166;
(c.) User closes doors 40, 42 and enters an access code on control panel 162;
(d.) User selects “Start Heat Cycle” on control panel 162;
(e.) Control system 160 verifies that doors 40, 42 are closed and locked via magnetic lock 178;
(f.) Heating system 250 turns on and the cycle countdown timer begins (in a preferred embodiment 90 minutes);
(g.) Heating system 250 remains on until the air temperature in the cabinet, as measured by internal sensor 172, achieves the setpoint (in the preferred embodiment 125° F.) for at least 15 seconds. Heating system 250 then remains off for 3 minutes;
(h.) When heat system 250 turns on again, it stays on until the air temperature in the cabinet achieves the setpoint (typically 125° F.) for at least 15 seconds;
(i.) The previous step repeats until the timer reaches the end of the heat treatment period, typically 90 minutes;
(j.) The control panel 162 then displays that the cycle is complete;
(k.) If the control system 160 detects that key cycle parameters were not met, control panel 162 notifies the user of the deficiency; and
(l.) The user can then enter an access code on control panel 162 to unlock doors 40, 42 and remove the treated items from chamber 25.

### Dry Cycle

(a.) User loads chamber 25 with items in to be treated;
(b.) User verifies that no person or other animal is inside chamber 25 and presses sweep button 166;
(c.) User closes doors 40, 42 and enters an access code on control panel 162;
(d.) User selects “Start Dry Cycle” on control panel 162;
(e.) Control system 160 verifies that doors 40, 42 are closed and locked via magnetic lock 178;
(f.) Control system 160 then opens inlet vent valve and outlet vent valve, activates exhaust fan, and activate heat system 250. The cycle countdown timer begins;
(g.) Heat system 250 remains on unless the air temperature in the cabinet exceeds the control setpoint (in the preferred embodiment 125° F.) for at least 15 seconds. Heat system 250 remains off for 3 minutes;
(h.) When heat system 250 turns on again, it stays on until the air temperature in the cabinet exceeds the setpoint (typically 125° F.) for at least 15 seconds;
(i.) The previous step repeats until the timer reaches the end of the selected drying period and then vent valves 53, 55 are closed;
(j.) Control panel 162 then shows that the cycle is complete; and the user can then enter an access code on control panel 162 to unlock doors 40, 42 and remove the treated items from chamber 25.

For convenience of the reader, following is a summary of parts and reference numbers in the written Specification and Drawings hereof:

| Part Number | Part Description |
|---|---|
| 10 | Preferred Embodiment |
| 20 | Housing Unit |
| 22 | Floor Member |
| 24 | Rear Wall |
| 25 | Chamber |
| 26 | First Side Wall |
| 28 | Second Side Wall |
| 30 | Upper Member |
| 40 | First Door |
| 42 | Second Door |
| 43 | Lip |
| 44 | Viewing Window |
| 45 | Handle |
| 48 | Hinges |
| 49 | First Inlet Fan |
| 50 | First Ventilated Port |
| 51 | Second Exhaust Fan |
| 52 | Second Ventilated Port |
| 53 | First Vent Valve |
| 54 | Third Ventilated Port |
| 55 | Second Vent Valve |
| 56 | Exterior Sensor Port |
| 60 | Ozone Generation System |
| 62 | Main Fan |
| 64 | UV Lamps |
| 65 | Ballasts |
| 66 | First Vents |
| 68 | Second Vents |
| 70 | Engine Encasement |
| 71 | Frame |
| 80 | Humidity Generation System |
| 82 | Water Tray |
| 84 | Lip |
| 86 | Inside Area |
| 87 | Wire |
| 88 | Piezo Mister |
| 90 | Plunger Mechanism |
| 92 | Spring-loaded Plunger |
| 93 | Head Portion |
| 94 | Bracket |
| 96 | Distal End |
| 97 | Through-holes |
| 98 | Intermediate Area |
| 99 | Air Duct |
| 100 | Corrugated Cage |
| 102 | Swinging Door |
| 104 | Hinged Joints |
| 106 | Top Member |
| 108 | 3-Sided Wall Member |
| 110 | Bottom Member |
| 112 | Shoulders |
| 114 | Through-holes |
| 116 | Inside Area |
| 118 | Base Plate |
| 119 | Ledge |
| 120 | Ozone Destruction System |
| 122 | Case |
| 124 | Destructor Fan |
| 125 | Inlet |
| 126 | Catalyst |
| 127 | Outlet |
| 160 | Control System |
| 162 | Control Panel |
| 163 | Exterior Emergency Stop Button |
| 164 | Main Power Switch |
| 165 | Interior Emergency Stop Button |
| 166 | Sweep Button |
| 167 | Power Regulator |
| 168 | Wire Bus |
| 169 | Wiring |
| 170 | PLC |
| 171 | Expansion Module |
| 172 | Internal Sensor |
| 174 | External Sensor |
| 178 | Magnetic Lock |
| 179 | Armature |
| 180 | Alternate Humidity Generation System |
| 182 | Water Pan |
| 183 | Fill Area |
| 184 | Divider |
| 186 | Wicking Filters |
| 188 | Shelf |
| 190 | Horizontal Member |
| 192 | Ledge |
| 200 | Alternate Embodiment |
| 220 | Single Door Housing Unit |
| 222 | Floor Member |
| 224 | Rear Wall |
| 226 | First Side Wall |
| 228 | Second Side Wall |
| 230 | Upper Member |
| 240 | Door |
| 244 | Viewing Window |
| 250 | Heating System |
| 252 | Heating Element |
| 254 | Blower |

Having, thus, described exemplary embodiments of the subject matter of the present disclosure, it is noted that the within disclosures are exemplary only, and that various other alternatives, adaptations, and modifications may be made within the scope and spirit of the present inventive subject matter. Accordingly, the present subject matter is not limited to the specific embodiments as illustrated herein, but is limited only by the following claims.

What is claimed:

1. An ozone sanitizing and disinfecting device comprising a closable chamber, an ozone generating device, a humidity generating system, a catalyst to convert ozone to oxygen, and a control system capable of maintaining a minimum level of relative humidity in the chamber between treatment cycles.

2. The device of claim 1 further comprising a sensor to measure the relative humidity in the surrounding environment and can predictively modify the treatment cycle parameters to ensure that the proper relative humidity is achieved inside the device during the treatment cycle resulting in a shorter treatment cycle.

3. The device of claim 1 further comprising a humidity generating system that is accessible, without the use of tools, by the user for periodic water replenishment and cleaning.

4. The device of claim 1 further comprising a humidity generating system with a water level indication that is easily viewable when the operator opens the chamber door(s).

5. The device of claim 1 further comprising a humidity generating system that uses a fan and wicking filters to evaporate water.

6. The device of claim 1 further comprising a humidity generating system that uses a piezoelectric transducer to atomize water.

7. The device of claim 1 further comprising a system to introduce ambient air from the surrounding environment into the chamber, after the treatment cycle, to ensure that the inside of the chamber is below the humidity level that allows mold to grow.

8. An ozone sanitizing and disinfecting device comprising a closable chamber, an ozone generating device, a humidity generating system, a catalyst to convert ozone to oxygen, and a control system capable of maintaining a minimum level of relative humidity in the chamber between treatment cycles, said ozone sanitizing and disinfecting device further comprising a sensor to measure the relative humidity in the surrounding environment and can predictively modify the treatment cycle parameters to ensure that the proper relative humidity is achieved inside the device during the treatment cycle resulting in a shorter treatment cycle.

9. The device of claim 8 further comprising a sensor to measure the relative humidity in the surrounding environment and can predictively modify the treatment cycle parameters to ensure that the proper relative humidity is achieved inside the device during the treatment cycle resulting in a shorter treatment cycle.

10. The device of claim 8 further comprising a humidity generating system that is accessible, without the use of tools, by the user for periodic water replenishment and cleaning.

11. The device of claim 8 further comprising a humidity generating system with a water level indication that is easily viewable when the operator opens the chamber door(s).

12. The device of claim 8 further comprising a humidity generating system that uses a fan and wicking filters to evaporate water.

13. The device of claim 8 further comprising a humidity generating system that uses a piezoelectric transducer to atomize water.

14. The device of claim 8 further comprising a system to introduce ambient air from the surrounding environment into the chamber, after the treatment cycle, to ensure that the inside of the chamber is below the humidity level that allows mold to grow.

15. A method of disinfecting and sanitizing objects, said method using a closable and lockable chamber having an ozone generation system, an ozone destruction system, and a humidity generation system, all in electronic communication with a control system, comprising the steps of:
a. placing at least one object to be disinfected and sanitized inside said closable chamber;
b. closing and locking said closable chamber;
c. activating, if needed, said humidity generation system via said control system to achieve a predetermined humidity level within said closable chamber;
d. activating said ozone generation system via said control system to achieve a predetermined ozone level within said closable chamber from ambient air for a predetermined period, wherein said ozonated air traverses the object(s) to be disinfected and sanitized;
e. activating said ozone destruction system via said control system to achieve a predetermined reduction in ozone level within said closable chamber;
f. unlocking said chamber via said control system.

16. The method of claim 15, wherein said closable and lockable chamber further has a heating system in electronic communication with said control system, further comprising the steps of:
activating said heating system via said control system to achieve a predetermined temperature within said chamber for a predetermined period.
